(19) United States
(12) Patent Application Publication
(10) Pub. No.: US 2025/0312596 A1
(43) Pub. Date: Oct. 9, 2025

(54) METHODS AND SYSTEMS TO REDUCE SYMPTOMS OF CEREBRAL PALSY IN CHILDREN

(71) Applicant: UNIVERSITY OF WASHINGTON, Seattle, WA (US)

(72) Inventors: Chet T. MORITZ, Seattle, WA (US); Katherine M. STEELE, Seattle, WA (US); Siddhi SHRIVASTAV, Seattle, WA (US); Charlotte CASKEY, Seattle, WA (US)

(73) Assignee: UNIVERSITY OF WASHINGTON, Seattle, WA (US)

(21) Appl. No.: 18/863,278

(22) PCT Filed: May 17, 2023
(86) PCT No.: PCT/US2023/022557

§ 371 Date: Nov. 5, 2024

Related U.S. Application Data
(60) Provisional application No. 63/343,829, filed on May 19, 2022.

(51) Int. Cl. A61N 1/36; A63B 22/02

(52) U.S. Cl. CPC A61N 1/36003; A61N 1/36034; A63B 22/02; A63B 2213/004; A63B 2214/00

(57) ABSTRACT

Methods and systems to reduce symptoms of cerebral palsy in children are disclosed herein. In one embodiment, a method for alleviating spasticity of a lower extremity caused by a brain injury includes attaching at least one electrode on a surface of patient's skin, proximate to patient's spinal cord. The method also includes subjecting the patient to a regimen of training that improves walking of the patient; applying a series of pulses to the at least one electrode while the patient is in training; and in response to applying the series of pulses to the at least one electrode, inducing stimulation in the spinal cord that alleviates spasticity of the lower extremity.

### CROSS-REFERENCE TO RELATED APPLICATION

This application claims the benefit of U.S. Provisional Application No. 63/343,829, filed May 19, 2022, the disclosure of which is expressly incorporated herein by reference in its entirety.

### BACKGROUND

Cerebral palsy (CP) is caused by damage to the developing brain, which results in spasticity and impaired movement. For example, individuals with cerebral palsy often have spasticity, altered movement, and reduced walking speed compared to their peers. Spasticity can be thought of as inappropriate contractions which in some cases manifest as contractions on opposite sides of the muscles, or on opposite side of joints, which leave an impression of the muscles fighting each other. Spasticity can be caused by inappropriate reflex responses where a muscle resists a stretch.

Conventional treatments include application of drugs or irreversible surgeries. For example, in some treatments certain nerves leading back from the affected muscles are selectively cut in an attempt to reduce the reflexes that are thought to cause the spasticity. Other conventional treatments include injecting muscles with botulinum toxin (e.g., Botox or similar) to paralyze the muscle. However, a downside of either of the above approaches is a possible permanent or temporary loss of function of the target muscle.

Accordingly, systems and methods are needed for improved treatments of cerebral palsy.

### SUMMARY

This summary is provided to introduce a selection of concepts in a simplified form that are further described below in the Detailed Description. This summary is not intended to identify key features of the claimed subject matter.

Briefly, the inventive technology is directed to alleviating spasticity and impaired movement caused by cerebral palsy (CP). The inventive technology is based on a combination of a rehabilitation program (e.g., a practice or activity) and a spinal cord stimulation that in combination alleviate spasticity of patient's lower extremities. In some embodiments, the practice or activity is walking on a treadmill. The spinal cord stimulation may be applied non-invasively through the skin, that is, transcutaneously. In other embodiments, surgically implanted electrodes may be used to induce stimulation to the spinal cord. In some embodiments, the electrodes are applied at the lower, thoracic lumbar cord. During the treatment, the intervals of transcutaneous spinal stimulation (tSCS) are combined with the intervals of locomotive training (i.e., practice or activity).

Combining the tSCS with training may alleviate spasticity by modulating spinal excitability and reducing spasticity. For example, short-burst interval locomotor treadmill training (SBLTT) consists of about 30-minutes treadmill exercise using 30 second alternating bouts of fast and slow walking. The treadmill training is supplemented with the spinal cord stimulation during the spinal cord stimulation (tSCS) phase of the training. In many cases, combining the SBLTT with the tSCS improves neuroplasticity in children with CP and may lead to a greater improvement than the training alone. In different embodiments, different wave forms may be used for such stimulation. For example, a base frequency may be in the range of 5-20 kHz, where the stimulation pattern repeats as a 1 ms pulse over each period of 33 ms. In other embodiments, other values of the stimulation frequency and/or pulse repetition may be used. In some embodiments, the amplitude of the stimulation pattern may be adjustable.

In one embodiment, a method for alleviating spasticity of a lower extremity caused by a brain injury includes: attaching at least one electrode on a surface of patient's skin.

proximate to patient's spinal cord; subjecting the patient to a regimen of training that improves walking of the patient; applying a series of pulses to the at least one electrode while the patient is in training; and in response to applying the series of pulses to the at least one electrode, inducing stimulation in the spinal cord that alleviates spasticity of the lower extremity.

In one aspect, the regimen of training comprises short-burst interval locomotor treadmill training (SBLTT) of about 30-minutes treadmill exercise.

In another aspect, the treadmill exercise comprises 30-seconds of alternating bouts of fast and slow walking.

In one aspect, the series of pulses are delivered as transcutaneous pulses (tSCS) to a lower back to the patient.

In one aspect, the series of pulses are delivered via two electrodes attached to the lower back of the patient.

In one aspect, the method also includes placing return electrodes over iliac crests of the patient or on a front of patient's hips or pelvis.

In one aspect, individual pulses are repeated at a frequency of about 30 Hz.

In one aspect, individual pulses are waveforms having a frequency in a range of 5-20 KHz.

In another aspect, individual pulses are waveforms having a frequency in a range of 10-180 KHz.

In one aspect, the frequency of waveforms is in a range of 5-10 KHz.

In one aspect, an amplitude of individual waveforms is in a range of 10-120 mA.

In one aspect, the waveforms are square waves.

In another aspect, the waveforms are sinusoidal waves.

In one aspect, the series of pulses are delivered through implanted electrodes.

In one embodiment, a system for alleviating spasticity of a lower extremity caused by a brain injury includes: at least one electrode configured for attachment on a surface of patient's skin, proximate to patient's spinal cord; and a controller configured for applying a series of pulses to the at least one electrode while the patient is subjected to a regimen of training that improves walking of the patient.

In one aspect, the regimen of training comprises short-burst interval locomotor treadmill training (SBLTT) of about 30-minutes treadmill exercise, and the treadmill exercise includes 30-seconds of alternating bouts of fast and slow walking.

In one aspect, the regimen of training comprises short-burst interval locomotor treadmill training (SBLTT) of about 90-minutes treadmill exercise.

In one aspect, the series of pulses are delivered as transcutaneous pulses (tSCS) of the patient via two electrodes attached to a lower back of the patient.

In one aspect, individual electrodes are 2 cm round electrodes attached proximate to T11 and L1 location of patient's spine.

In another aspect, the system includesreturn electrodes that are placed over iliac crests of the patient or on a front of patient's hips or pelvis.

In one aspect, individual pulses are repeated at a frequency of about 30 Hz.

In one aspect, individual pulses are rectangular pulses having a duration (T) of 1 ms.

In one aspect, individual pulses include waveforms having a frequency in a range of 5-20 KHz or in a range of 10-180 KHz.

In one aspect, the frequency of the waveforms is 10 KHz.

In another aspect, an amplitude of the waveforms is in a range of 10-120 m.A.

In one aspect, the waveforms are square waves or sinusoidal waves.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, where:

FIG. 1 is an illustration of a patient being instrumented with a stimulation apparatus in accordance with an embodiment of the present technology;

FIG. 2 is a graph of spinal stimulation signal in accordance with an embodiment of the present technology;

FIG. 3 is a diagram illustrating a method for alleviating cerebral palsy (CP) in accordance with an embodiment of the present technology;

FIGS. 4A-4D are graphs of lower extremity modified Ashworth scale (MAS) results in accordance with an embodiment of the present technology;

FIGS. SA-5D are graphs of pre-intervention and post-intervention walking distances in accordance with an embodiment of the present technology;

FIGS. 6A and 6B are graphs of ten-meter walk test trajectory for self-selected and fast-walking speeds in accordance with an embodiment of the present technology;

FIG. 7 is a graph of spasticity before and after treatment in accordance with an embodiment of the present technology;

FIG. 8 is a graph of spasticity trajectory across intervention phases (MAS) in accordance with an embodiment of the present technology; and

FIG. 9 is a graph of spasticity pre-and post-interventions (MAS) in accordance with an embodiment of the present technology.

### DETAILED DESCRIPTION

In order to better understand the technical solutions of the present disclosure, the embodiments of the present disclosure are described in detail below with reference to the accompanying drawings. It should be clear that the described embodiments are only a part of the embodiments of the present disclosure, but not all of the embodiments. Based on the embodiments in the present disclosure, other embodiments obtained by those of ordinary skill in the art fall within the protection scope of the present disclosure.

The terms used in the embodiments of the present disclosure are only for the purpose of describing specific embodiments, and are not intended to limit the present disclosure. As used in the embodiments of this application and the appended claims, the singular forms “a,” “the,” and “the” are intended to include the plural forms as well, unless the context clearly dictates otherwise.

It should be understood that the term “and/or” used in this document is only an association relationship to describe the associated objects, indicating that there can be three relationships, for example, A and/or B, which can indicate that A alone, A and B, and B alone. The character “/” in the present description generally indicates that the related objects are an “or” relationship.

It should be understood that although the terms ‘first’, ‘second’, and ‘third’ can be used in the present disclosure to describe thin film transistors, these thin film transistors should not be limited to these terms. These terms are used only to distinguish the thin film transistors from each other.

In the context of this disclosure, the terms “about,” “approximately,” “generally” and similar mean +/−5% of the stated value.

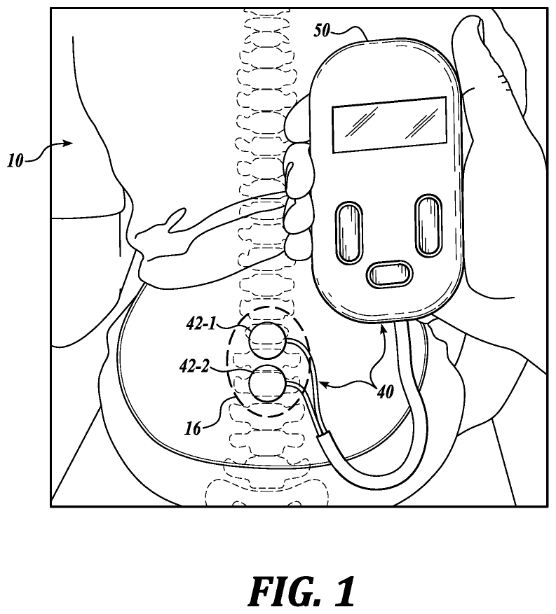

FIG. 1 is an illustration of a patient 10 being instrumented with a stimulation apparatus 40 in accordance with an embodiment of the present technology. In some embodiments, electrodes 42 (e.g., electrodes 42-1 and 42-2) are attached to skin surface of a treatment area 16 of the lower back of the patient, i.e., proximate to the spinal cord of the patient. A controller 50 (e.g., computer, mobile phone, tablet, game console, analog controller, etc.) controls application of the stimulus signal to the electrodes. A non-limiting example of such controller 50 is a SCONE neuromodulation device by SpineX Inc., Los Angeles, California. In operation, an operator (e.g., physician, physical therapist, trainer, etc.) may use the controller 50 to select the stimulation signal and/or to change the parameters of the stimulation signal. In some embodiments, the electrodes 42-1 and 42-2 may be 2 cm round electrodes attached to the lower thoracic and lumbar spine of the patient 10, for example, over the T11 and L1 vertebrae, or, in general, proximate to the patient's spinal cord. Return electrodes (e.g., two 5×10 cm rectangular electrodes) may be placed symmetrically over the iliac crests of the patient or on the front of the hips/pelvis. When the controller 50 applies a selected stimulus signal (e.g., a sequence of electrical excitation), patient's spine is stimulated by a transcutaneous transmission of the electrical signal to patient's spine. In different embodiments, the electrodes 42 may be implantable electrodes.

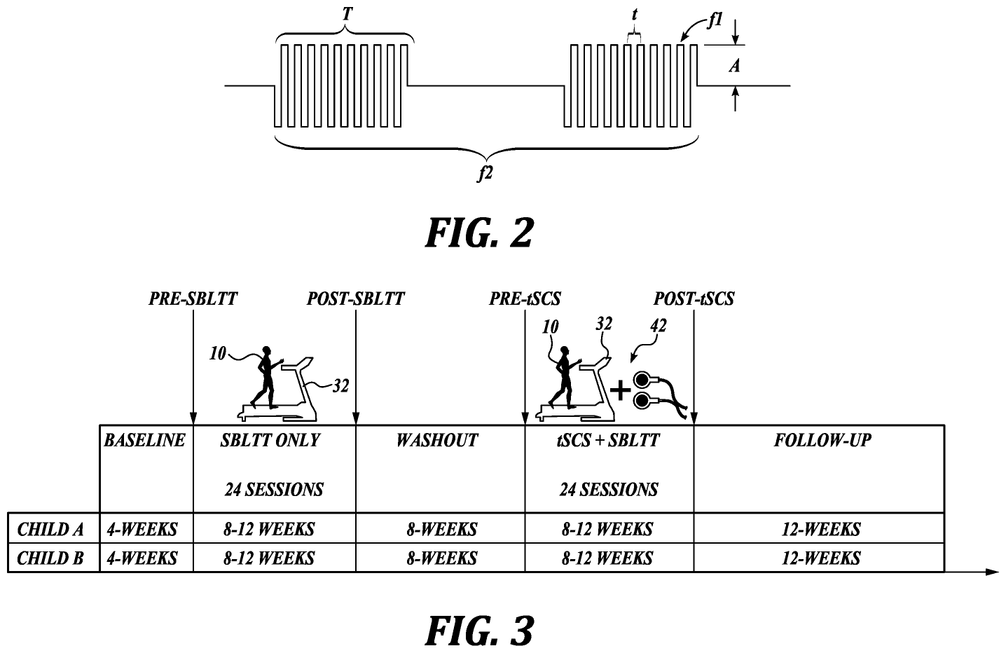

FIG. 2 is a graph of spinal stimulation signal in accordance with an embodiment of the present technology. The illustrated stimulation pattern (also referred to as the stimulation waveform) is delivered as a series of pulses, each of duration T that induce stimulation in the spinal cord that, in turn, alleviates spasticity of the lower extremity. Individual stimulation signals are characterized by their frequency f1, period t and amplitude A, each of these parameters being controllable by the controller 50. The stimulation signals are illustrated as square waves, however in different embodiments the stimulation signals may be sinusoidal or other waves. The pulses of signals may repeat at frequency f2. Therefore, in the illustrated embodiments, the stimulation signal is applied in an on-off manner at a selected duty factor.

As a non-limiting example, the stimulation waveform may use a biphasic, rectangular pulses with a duration T=1 ms and frequency of f2=30 Hz. Each pulse may be filled with a waveform having a carrier frequency f1 in the range of 5-10, 5-20 or 10-180 KHz. The stimulation may be delivered to each of the electrodes 42-1, 42-2 at a waveform amplitude of, for example, 20-60 mA or 10-120 mA for a duration of 90 minutes by an operator (e.g., a trained physical therapist, or a researcher) during the sessions that include both tSCS and SBLTT segments as described in more details below. In different embodiments, stimulation pulses (e.g., pulses with a duration T and frequency f2) may be delivered continuously during the tSCS+SBLTT regimen, or the stimulation pulses may be delivered on and off during this regimen.

Stimulation intensity (e.g., amplitude and duty factor of the stimulation waveform) may be determined by observation of improvements in patients' walking, such as increased knee flexion and dorsiflexion during walking and also by the patients' self-report of sensation of the stimulation. Without being tied to theory, it is believed that the stimulation waveforms at around 10 kHz or within a range of 5-10 kHz effectively induce blocking c-fibers in the skin, allowing more current (i.e., higher amplitude A of the waveform) to be comfortably applied to the skin of the patient. It is further believed that tSCS leads to preferential activation of the la afferents, thereby depolarizing the motor neurons without necessarily causing direct muscle contractions. The tSCS may also alter the excitability of intra-spinal neuronal networks, possibly by augmenting pre-and post-synaptic inhibitory mechanisms, and therefore may be a viable alternative to pharmacological and surgical approaches to manage spasticity. The combination of tSCS and physical training reduces spasticity and improves function in people with spinal cord injury, as further explained below.

FIG. 3 is a diagram illustrating a method for alleviating cerebral palsy (CP) in accordance with an embodiment of the present technology. The test subjects (child A and child B) are 12- and 4-years old males with spastic CP. Each completed 24-sessions of two interventions: short-burst interval locomotor treadmill training (SBLTT) only and SBLTT combined with spinal stimulation (tSCS). Child A completed all the interventions in a laboratory setting. Child B partook in an integrated home program in which a trained physical therapist led intervention sessions in his home. Some intervention sessions and all assessment sessions took place in a laboratory setting. There was a washout period between the two interventions (also referred to as the phases) to account for carry-over effects, and a 3-month follow-up after the second intervention. A person of ordinary skill would understand that the above-quoted time periods are exemplary only, and that different time periods may apply in different embodiments.

In some embodiments, during the SBLTT only phase, each intervention session included a 5-20 minute active warm-up, and a 30-minute of high-intensity SBLTT, followed by a 5-minute active cool down. Rest breaks were provided as needed. SBLTT is a form of treadmill training and included alternating 30-second bursts of slow and fast walking speeds. The treadmill starting speeds were determined as 80-100% of children's overground 10-meter walking speeds. The fast speeds progressed within and between the intervention sessions, while the slow speeds remained constant. Children's total training time each session was based on age, cardiovascular endurance, functional status and abilities.

During the tSCS+SBLTT phase, the same sequence of training, including warm-up and cool-down, was repeated with the addition of tSCS while the participants are training. The stimulation was delivered at an amplitude of 20 to 60 mA for a maximum of 90 minutes by a trained interventionist and researcher during all tSCS+SBLTT intervention sessions for both children.

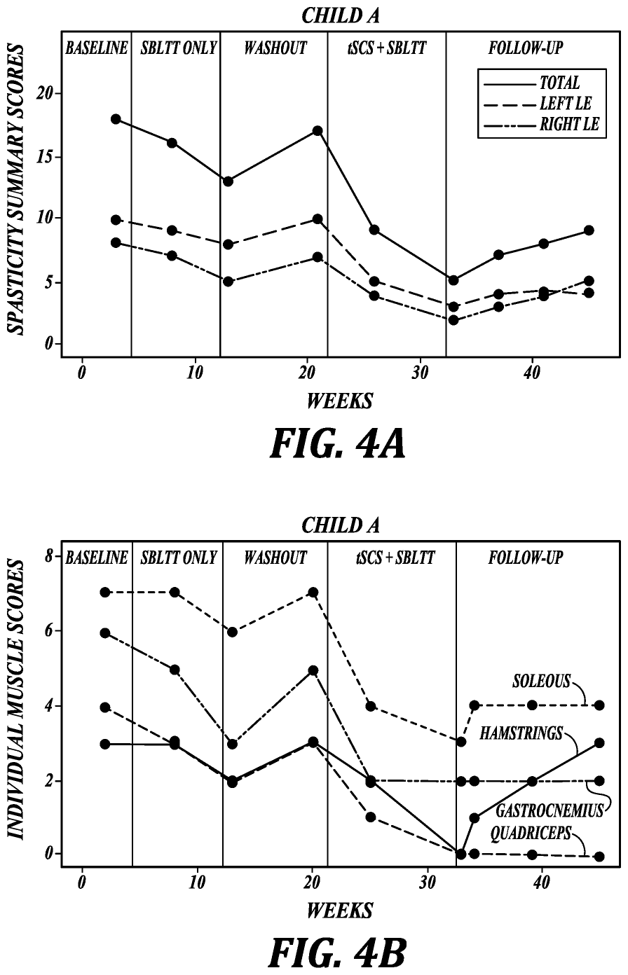

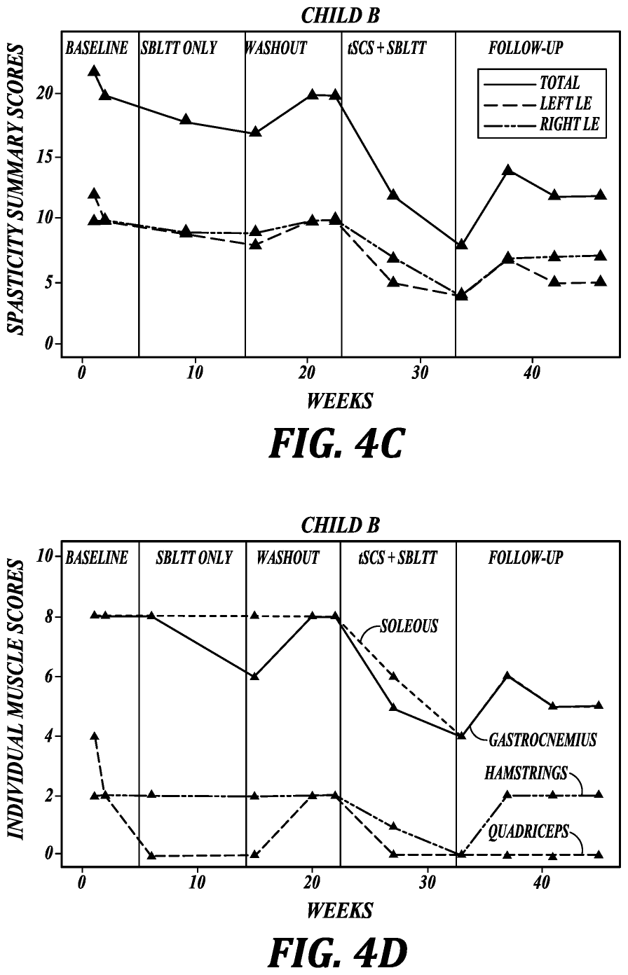

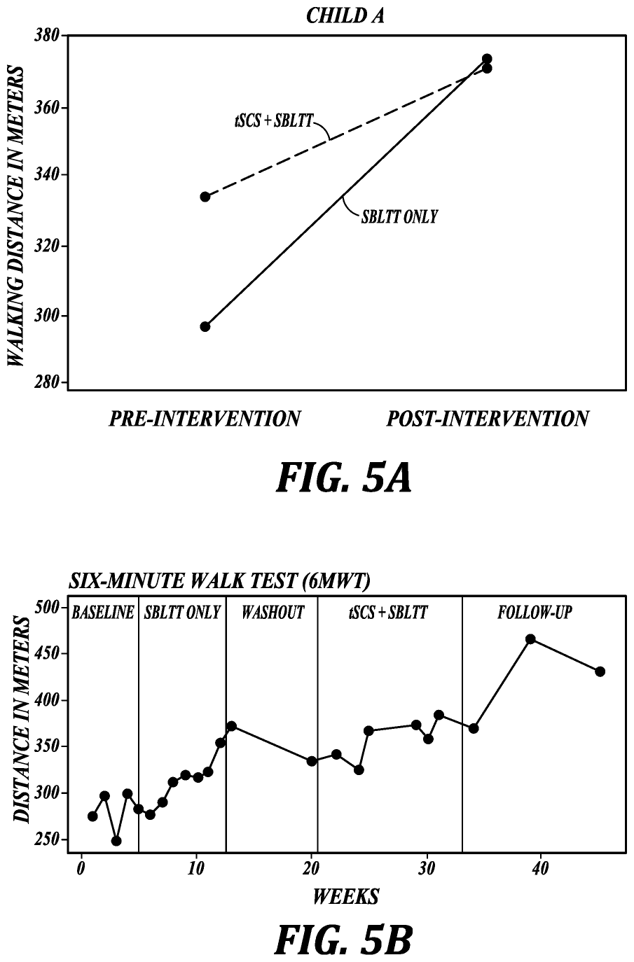

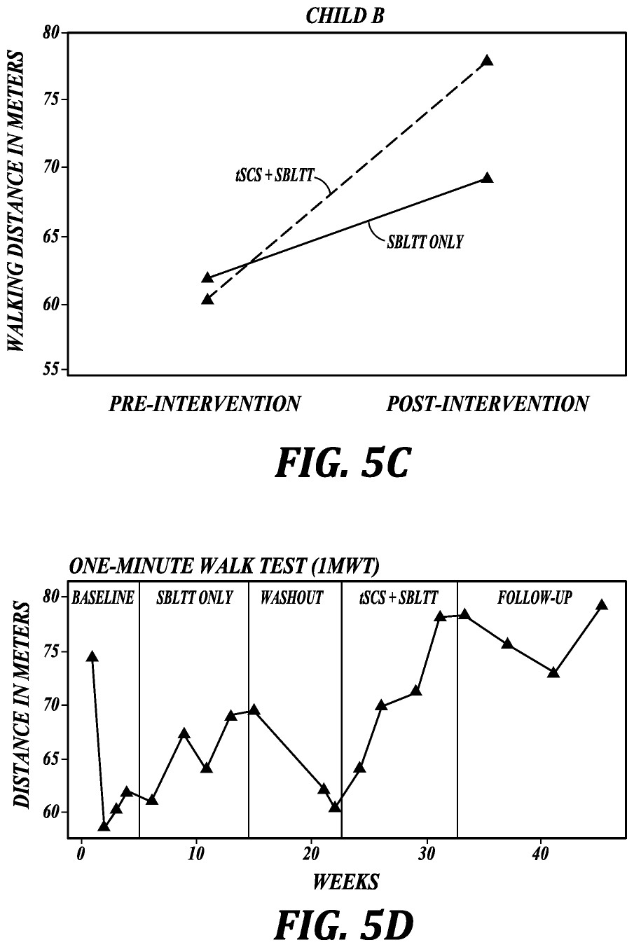

FIGS. 4A-4D are graphs of lower extremity modified Ashworth scale (MAS) results in accordance with an embodiment of the present technology. For each graph, the horizontal axis shows time in weeks, with different segments of the regimen corresponding to the segments shown in FIG. 3. The vertical axis indicates spasticity summary scores as MAS scores for child A (FIGS. 4A and 4B) and child B (FIGS. 4C and 4D). The graphs in FIGS. 4A and 4C include total MAS results, left lower extremity (LE) and right LE results for combined muscles. The graphs in FIGS. 4B and 4D represent MAS results for different muscle groups

FIG. 3 is a diagram illustrating a method for alleviating cerebral palsy (CP) in accordance with an embodiment of the present technology. The test subjects (child A and child B) are 12- and 4-years old males with spastic CP. Each completed 24-sessions of two interventions: short-burst interval locomotor treadmill training (SBLTT) only and SBLTT combined with spinal stimulation (tSCS). Child A completed all the interventions in a laboratory setting. Child B partook in an integrated home program in which a trained physical therapist led intervention sessions in his home. Some intervention sessions and all assessment sessions took place in a laboratory setting. There was a washout period between the two interventions (also referred to as the phases) to account for carry-over effects, and a 3-month follow-up after the second intervention. A person of ordinary skill would understand that the above-quoted time periods are exemplary only, and that different time periods may apply in different embodiments.

Spasticity improved in all muscles during the tSCS and SBLTT for both children, and largely persisted during the three months of the follow-up period. The greatest improvements in spasticity were observed in the gastrocnemius (Child B) and soleus in both children following the combination of tSCS and SBLTT phase (FIGS. 4B and 4D).

The hamstrings were the only muscle group to return to baseline spasticity during follow-up.

FIGS. 5A-SD are graphs of pre-intervention and post-intervention walking distances in accordance with an embodiment of the present technology. The horizontal axes in graphs of FIGS. 5A and 5C represent different phases of the treatment, and the horizontal axes in graph of FIGS. 5B and 5D represent time spent in different phases of the treatment. The vertical axis represents walking distance in meters. Figures SA and SB illustrate results for child A, and FIGS. 5C and 5D illustrate results for child B. FIG. 5B illustrates a six-minute test for child A, and FIG. 5D illustrates a one-minute test for child B.

As shown in FIGS. 5A and 5C, walking distances improved for both children during both treatments (SBLTT only, and combined tSCS and SBLTT). As shown in FIGS. 5B and 5D, the longest walking distances were observed at the end of the combined tSCS and SBLTT phase (treatment), and during the follow-up. For example, for the six-minute walking test of the child A, the walking distances were respectively 25% and 27% longer after SBLTT only phase and after the combined tSCS and SBLTT phase, whereas for the one-minute walking test of the child B the walking distances were 13% and 27% longer as compared to before each treatment. The walking distances remained longer for both children during the follow-up period.

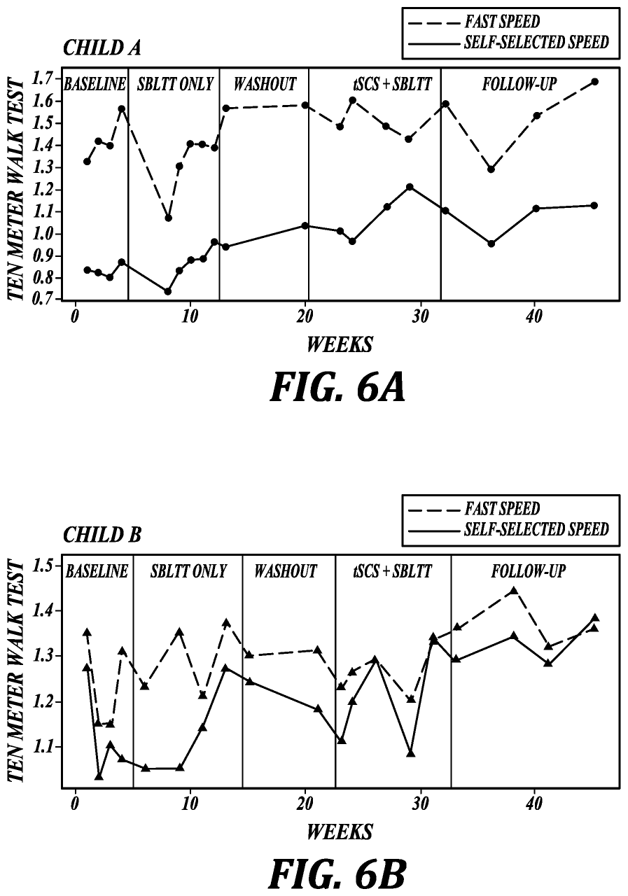

FIGS. 6A and 6B are graphs of ten-meter walk test trajectory for self-selected and fast-walking speeds in accordance with an embodiment of the present technology. FIG. 6A shows results for child A, and FIG. 6B shows results for child B. The horizontal axis shows the time scale divided into different phases of the treatment. The vertical axis shows the walking speed in meters per second. The two curves in each graph correspond to the self-selected speed and the fast speed that each child can achieve at different phases of the treatment.

In each case, the self-selected walking speeds improved for both children during the SBLTT only phase and remained higher during the combined tSCS and SBLTT phase in both children. The fast walking speeds were more variable in both children. For instance, child A walked at a self-selected speed of approximately 1.2 m/s during the combined tSCS and SBLTT phase, which was greater than his self-selected speed of 1.0 m/s after the SBLTT only phase. This speed was maintained at the end of the follow-up. Child B walked at a faster self-selected walking speeds in both the combined tSCS and SBLTT phase and the SBLTT only phase, reaching a speed of 1.3 m/s at the end of the combined tSCS and SBLTT phase, and 1.4 m/s during the follow-up. Child B had high variability of fast walking speeds throughout the study, likely due to his younger age, which may also explain why his self-selected and fast walking speeds were closer to each other.

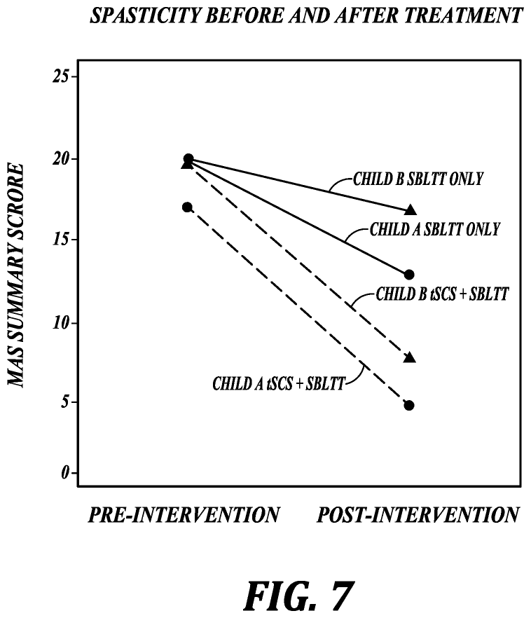

FIG. 7 is a graph of spasticity before and after treatment in accordance with an embodiment of the present technology. The horizontal axis shows time divided into the pre-intervention and post-intervention segments. The vertical axis shows MAS summary score. Different curves correspond to the same two subjects, each subject having his scores for the combined tSCS and SBLTT phase, and for the SBLTT phase alone. The pre-intervention scores correspond to the end of baseline (for the SBLTT only phase) and the end of washout (for the combined tSCS and SBLTT phase). The post-intervention scores correspond to the beginning of washout (for the SBLTT only phase) and beginning of follow-up (for the combined tSCS and SBLTT phase). The total spasticity summary score combines both legs.

As can be seen from the graphs, the summary score is reduced by about 12 points after the combined tSCS and SBLTT phase for both children. Furthermore, the treatment with the combined tSCS and SBLTT phase resulted in greater improvements in spasticity than the SBLTT phase alone for both children. For example, the spasticity summed across both extremities is reduced by about 5 points on average during the SBLTT only phase.

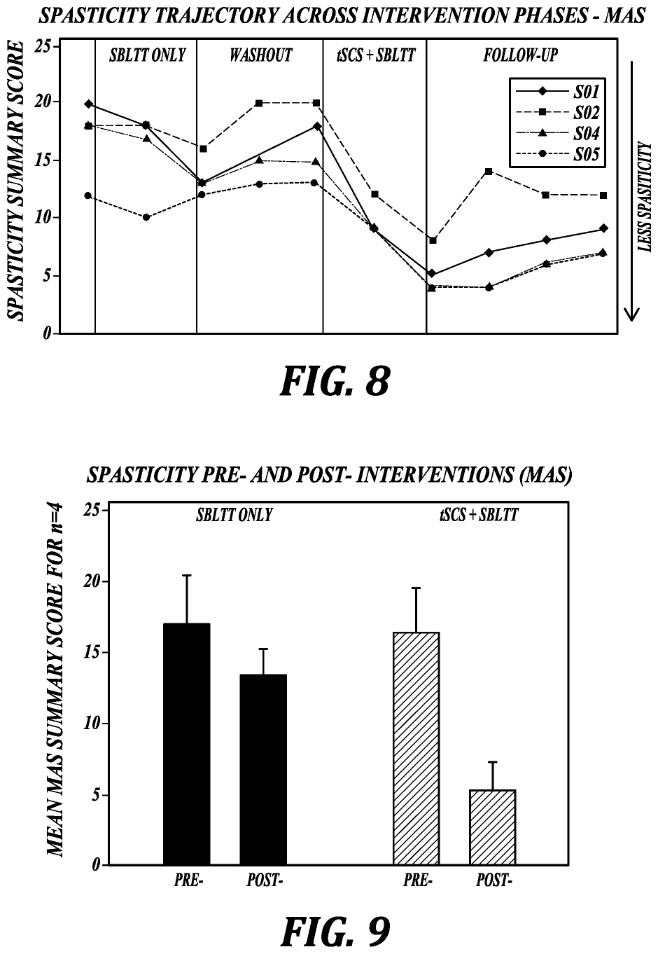

FIG. 8 is a graph of spasticity trajectory across intervention phases (MAS) in accordance with an embodiment of the present technology. This graph is comparable to the graphs shown in FIGS. 4A and 4C for the total spasticity, the difference being that FIG. 8 includes a larger number of patients (four). For each graph, the horizontal axis shows time in weeks, with different segments corresponding to different phases of the treatment. The vertical axis indicates spasticity summary scores as MAS scores for four patients (S01, S02, S03 and S04) for combined muscles.

As can be seen from the graphs, the summary score (MAS) is reduced by about 10-15 points after the combined tSCS and SBLTT phase for each patient (in comparison to the beginning of the SBLTT phase). Furthermore, the treatment with the combined tSCS and SBLTT phase resulted in greater improvements in spasticity than the SBLTT phase alone for all patients by about 7-10 points.

FIG. 9 is a graph of spasticity pre-and post-treatment (MAS) in accordance with an embodiment of the present technology. The test population again included four patients, like in FIG. 8. The bars of the graph represent the averaged pre-and post-treatment spasticity results when the treatment includes the SBLTT only phase (solid bars) and when the treatment includes the combined tSCS and SBLTT phase (hatched bars), the post-treatment spasticity results being improved in both cases. However, while average spasticity is improved by about 4 points for the treatment that includes the SBLTT only phase (solid bars), average spasticity is improved by about 10 points when the treatment includes the combined tSCS and SBLTT phase (hatched bars).

Many embodiments of the technology described above may take the form of computer-or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above.

Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

What is claimed is:

1. A method for alleviating spasticity of a lower extremity caused by a brain injury, comprising:
attaching at least one electrode on a surface of patient's skin, proximate to patient's spinal cord;
subjecting the patient to a regimen of training that improves walking of the patient;
applying a series of pulses to the at least one electrode while the patient is in training; and
in response to applying the series of pulses to the at least one electrode, inducing stimulation in the spinal cord that alleviates spasticity of the lower extremity.

2. The method of claim 1, wherein the regimen of training comprises short-burst interval locomotor treadmill training (SBLTT) of about 30-minutes treadmill exercise.

3. The method of claim 2, wherein the treadmill exercise comprises 30-seconds of alternating bouts of fast and slow walking.

4. The method of claim 2, wherein the series of pulses are delivered as transcutaneous pulses (ISCS) to a lower back to the patient.

5. The method of claim 4, wherein the series of pulses are delivered via two electrodes attached to the lower back of the patient.

6. The method of claim 5, further comprising placing return electrodes over iliac crests of the patient or on a front of patient's hips or pelvis.

7. The method of claim 4, wherein individual pulses are repeated at a frequency of about 30 Hz.

8. The method of claim 4, wherein individual pulses comprise waveforms having a frequency in a range of 5-20 KHz.

9. The method of claim 4, wherein individual pulses comprise waveforms having a frequency in a range of 10-180 KHz.

10. The method of claim 8, wherein the frequency of waveforms is in a range of 5-10 KHz.

11. The method of claim 8, wherein an amplitude of individual waveforms is in a range of 10-120 mA.

12. The method of claim 8, wherein the waveforms are square waves.

13. The method of claim 8, wherein the waveforms are sinusoidal waves.

14. The method of claim 2, wherein the series of pulses are delivered through implanted electrodes.

15. A system for alleviating spasticity of a lower extremity caused by a brain injury, the system comprising:
at least one electrode configured for attachment on a surface of patient's skin, proximate to patient's spinal cord; and
a controller configured for applying a series of pulses to the at least one electrode while the patient is subjected to a regimen of training that improves walking of the patient.

16. The system of claim 15, wherein the regimen of training comprises short-burst interval locomotor treadmill training (SBLTT) of about 30-minutes treadmill exercise, and wherein the treadmill exercise comprises 30-seconds of alternating bouts of fast and slow walking.

17. The system of claim 15, wherein the regimen of training comprises short-burst interval locomotor treadmill training (SBLTT) of about 90-minutes treadmill exercise.

18. The system of claim 15, wherein the series of pulses are delivered as transcutaneous pulses (ISCS) of the patient via two electrodes attached to a lower back of the patient.

19. The system of claim 18, wherein individual electrodes are 2 cm round electrodes attached proximate to T11 and L1 location of patient's spine.

20. The system of claim 19, further comprising return electrodes that are placed over iliac crests of the patient or on a front of patient's hips or pelvis.

21. The system of claim 15, wherein individual pulses are repeated at a frequency of about 30 Hz.

22. The system of claim 15, wherein individual pulses are rectangular pulses having a duration (T) of 1 ms.

23. The system of claim 15, wherein individual pulses comprise waveforms having a frequency in a range of 5-20 KHz or in a range of 10-180 KHz.

24. The system of claim 23, wherein the frequency of the waveforms is 10 kHz.

25. The system of claim 23, wherein an amplitude of the waveforms is in a range of 10-120 mA.

26. The system of claim 22, wherein the waveforms are square waves or sinusoidal waves.
